(19) United States
(12) Patent Application Publication
(10) Pub. No.: US 2013/0289498 A1
(43) Pub. Date: Oct. 31, 2013

(54) Catheter and Method for Making a Catheter

(71) Applicant: Gjalt BOSMA, Leeuwarden (NL)

(72) Inventor: Gjalt BOSMA, Leeuwarden (NL)

(73) Assignee: PENDRACARE INTERNATIONAL B.V., Leek (NL)

(21) Appl. No.: 13/459,975

(22) Filed: Apr. 30, 2012

(51) Int. Cl. A61L 29/14; B05D 7/00

(52) U.S. Cl. 604/265; 427/2.3

(57) ABSTRACT

Disclosed is a catheter and a method for making a catheter. The catheter has a proximal end and a distal end, and includes from the distal end towards the proximal end a distal section, an intermediate section and a proximal section, wherein the intermediate section is provided with a coating such that the intermediate section experiences less friction in use than the distal section and the proximal section.

### BACKGROUND OF THE INVENTION

1) Field of the Invention

The present invention relates to a catheter that can be inserted into a body lumen of animals and/or humans, such as into a vascular system. Such catheters include guiding and/or diagnostic catheters for guiding and diagnostic purposes, respectively. The catheter can be applied in the field of cardiology, radiology, oncology and neurology, for example.

2) Prior Art

It is known in the art to provide catheters with a coating to reduce friction between the catheter and the body lumen. However, a drawback of this low friction is that the catheter easily slips out of the body lumen during the introduction of the catheter therein. Furthermore, other unintentional movements of the catheter occur.

The object of the present invention is to reduce the above problems and to provide a catheter wherein unintentional movement in use is reduced.

### SUMMARY OF THE INVENTION

This object is achieved with the catheter according to the present invention, the catheter having a proximal end and a distal end, and comprising from distal end towards the proximal end:
a distal section;
an intermediate section; and
a proximal section,
   wherein the intermediate section is provided with a coating such that the intermediate section experiences less friction in use than the distal section and the proximal section.

According to the invention, the intermediate section is coated, while the distal section and proximal section are uncoated or coated with a different coating.

Due to the difference in coating of the distal section and the intermediate section, the distal section experience a higher friction in use, e.g. when the catheter is introduced in a vascular system, relative to the intermediate part. Therefore, unintentional movement of the catheter during the procedure is prevented. Especially during introduction of the catheter into a body lumen such as a vessel, the relatively high friction of the distal section prevents the catheter from slipping out the vessel during the introduction thereof. Optionally, the friction of the distal section is further increased and also higher as compared to the friction of the proximal section. This can be achieved by applying a different coating for the distal sections for example.

Furthermore, the distal section provides additional passive backup support. For example, the distal section provides good grip at the ostium and support of the opposite aorta wall. Also during a procedure, the distal section prevents unintentional movement of the catheter.

The proximal section also has a higher friction in use than the intermediate section, for example because it is uncoated or coated with a different coating than the intermediate section. The proximal section will in general stay outside the body and be used for manipulation of the catheter. Therefore, the proximal section of the catheter according to the invention provides more grip which enables easy manipulation of the catheter.

For example the coating comprises Teflon. Furthermore, other low friction materials, even those normally not considered as coating materials, may be used.

In a preferred embodiment of the invention the coating is a hydrophilic coating. A hydrophilic coating allows for a smooth catheter introduction and results in a low friction during catheter advancement, especially in tortuous and/or calcified anatomy. Furthermore, it leads to a reduced vessel spasm in radial approach resulting in more patient comfort. Moreover, providing a hydrophilic coating causes less trauma to the vessel wall reducing the risk of debris flowing downstream which could lead to TIAs. In other words, the beneficial effects of using a hydrophilic coating are combined with good maneuverability in the catheter according to the invention. For example, the hydrophilic coating is a so called comfort coating as provided by DSM. The coating preferably is a hemocompatible antimicrobial coating.

In a preferred embodiment according to the invention the distal section has a length of 1-15 cm, preferably 3-15 cm, more preferably 3-10, even more preferably 5-9 cm and most preferably approximately 7 cm.

In a preferred embodiment according to the invention the proximal section has a length of at least 3 cm, preferably at least 5 cm, more preferably at least 10 cm, even more preferably at least 20 cm and most preferably at least 25 cm.

Preferably, the proximal section forms at least a part of the section of the catheter which in use stays outside the body.

Other lengths are also possible for the distal section and/or the proximal section. In particular, the length can be selected according to the application of the catheter.

Preferably, the catheter according to the invention is a guiding and/or diagnostic catheter.

In a preferred embodiment of the invention the catheter comprises a tip comprising in the direction of the distal end towards the proximal end;
a soft tip;
adjacent to the soft tip an intermediate tip; and
separated from the soft tip by the intermediate tip a distal tip,
   wherein the tips are provided with different characteristics relating to the resistance against bending and/or positioning and/or relaxation of the catheter in a body lumen.

The tip of the catheter, i.e. a distal part of the catheter, is provided with at least three tip regions: a soft tip at the outer end of the tip, adjacent thereto an intermediate tip, and adjacent thereto towards the proximal end of the catheter a distal tip. The tips are provided with different characteristics. The tip of the catheter can be tuned to minimize resistance against bending and/or positioning and/or relaxation of the catheter in a vascular system. These movements also include rotating and/or sliding of the catheter in the vascular system. By minimizing the resistance moving the catheter in a vascular system is made easier. Additionally, the damage to the vascular system due to the movement of the catheter is also minimized.

By applying these at least three tip regions in combination with the sections having different frictions a further user-friendly catheter is achieved that both moves relatively smooth at the tip and prevents unintentional movements of the different sections as compared to conventional catheters.

It is understood that the distal tip may have the same characteristics as the body of the catheter, i.e. the catheter has a body with first characteristics, an intermediate tip with second characteristics and a soft tip with third characteristics, e.g. the body forms the distal tip.

Preferably, in an embodiment of the catheter according to the present invention the tip of the catheter gradually becomes softer towards the soft tip end. The relatively soft tip at the outer end of the tip is very flexible to enable alignment with the coronary take-off and to achieve an a-traumatic behaviour of the catheter according to the present invention. The intermediate tip is provided with sufficient stiffness. In use, this intermediate tip realizes a high backup force. This enables, for example, keeping the tip in the ostium when a force is applied to the catheter during an interventional procedure. This is especially relevant for guiding catheters. The distal tip, the region of the catheter tip the closest to the proximal end of the catheter, is less important for tip alignment and a-traumatic behaviour. Therefore, this distal tip is relatively stiff to enable applying relatively high pushing forces and torques, together with a low resistance during catheter movement.

Preferably, the distal tip is made of a relatively very elastic material as compared to the intermediate and soft tips to improve the kink resistance. By providing the different regions of the catheter tip with different characteristics the backup support and/or the alignment in the ostium is improved including the placement of the tip in a vascular system. In addition, damage to such system is minimised.

The catheter according to the invention is specifically advantageous for so-called guiding and diagnostic catheters as it improves positioning of the catheter and minimizes the risk of damaging the vascular system, for example.

Preferably, the catheter according to the invention comprises an elongate tube comprising:
at least one plastic inner layer;
at least one reinforcement layer; and
at least one plastic top layer.

Preferably, the soft tip comprises a top layer of a soft material, more preferably a soft polyamide. The use of this soft material has proven to result in a very flexible tip and to minimize damage to the vascular system when moving the catheter. A soft polyamide that can be used for the catheter according to the present invention is PEBAX®. Preferably, the intermediate tip and/or the distal tip comprise a rigid polymer. More preferably, the rigid polymer comprises fillers, most preferably polyamide (Vestamid®). Alternatively, polyamide and/or PEBAX® is used.

Preferably, the top layer of the soft tip and other tips comprise the materials mentioned above. According to the invention it is also possible for the top layer of the individual regions to only consist of the materials mentioned above.

Preferably, in an embodiment of the catheter according to the present invention the distal tip has a length in the range of 5-20 cm, preferably 10-15 cm and most preferably has a length of about 10 cm. Experiments have shown that most preferably a length of 10 cm provides the highest maneuverability of the catheter during movement of the catheter in a vascular system, and at the same time minimizes damage to the vascular system due to these movements. Specific dimensions of the catheter depend on the required backup support and/or the (local) anatomic shape.

It is noted that the catheter tip is not necessarily equal to the distal section as defined above. For example, the coating of the intermediate section may partly cover the catheter tip.

In an advantageous preferred embodiment of the catheter according to the present invention the reinforcement layer comprises so-called braiding and the top layer extends between the braiding.

By providing top layer material between the braiding the characteristics of the catheter are improved. In known catheters material between the braiding does not origin from the top layer. In the catheter according to the invention this material is provided between the braiding using a heat shrink or pressure, for example.

The present invention also relates to a method of producing a catheter, comprising the step of providing a coating to an intermediate section of a catheter between an adjacent distal section and an adjacent proximal section of the catheter such that the intermediate section experiences less friction in use than the distal section and the proximal section.

Such a method provides the same effects and/or advantages as mentioned above for the catheter according to the invention.

In a preferred embodiment, the method comprises the steps of:
providing the catheter with an elongate tube;
at a tip of the catheter locally removing material from and/or preventing material to be provided on the top layer of the tube;
replacing the removed material with, and/or placing, a different material.

Preferably, the top layer is locally removed at the catheter tip. More preferably, material is also removed from between the braiding of the reinforcement layer. For the purpose of this application removing material includes removal of material as well as the prevention of material being provided during the production of the catheter according to the present invention.

Preferably, the entire top layer is removed with the method according to the present invention. Removal of the top layer of an already produced tube can be achieved by etching, laser ablation, water jets, dissolving material, or by an appropriate heat treatment, for example.

In a preferred embodiment according to the present invention replacing the removed material includes providing a heat shrink tubing, injection molding and/or dipping to locally define the catheter characteristics.

By removing material and providing a heat and shrink tubing the characteristics, like stiffness of the region, can be defined locally depending on the function of this specific region of the catheter. This enables providing a dedicated catheter. Alternative ways to replace the material according to the present invention include the use of a dipping process, evaporation of fluid of a dissolved plastic, and injection moulding, for example. Also, other processes to replace material can be used, using pressure, for example. Preferably, the material of the top layer extends between the braiding of the reinforcement layer. This shows a relevant improvement of the characteristics of the catheter that is achieved.

In a preferred embodiment according to the present invention during locally removing material from the top layer slots are provided that are filled, when replacing the removed material, with a different material.

By providing slots it is possible to design the characteristics of this region of this catheter tip relating to the desired characteristics, like stiffness and flexibility. The amount, dimensions and/or orientation of the slots are relevant for the characteristics of this region and should be designed depending on the purpose. Slots can be provided by etching and/or by using mechanical techniques, such as water jets and/or punch rod. Preferably, the diameter or size of the slots is smaller than the size of the braiding wires in the reinforcement layer.

Alternatively, or in combination with the above approach, when removing material from the outer or top layer some parts of the former material are kept. Next, the final top layer material is provided. The resulting catheter according to the invention comprises alternately relatively hard and relatively soft parts. Preferably, over the entire outer surface the final top layer material is provided.

In a preferred embodiment according to the present invention replacing the removed materials involves providing cross linkers which can be activated to change catheter characteristics.

By activating the cross linkers the flexibility of the region provided with the cross linkers and the top coat material will change. The activation step can be provided using UV light, laser, X-ray or by using a chemical source, for example.

In a further preferred embodiment according to the present invention during replacing the removed material hard and soft areas are provided.

By providing hard and soft areas in the top layer of a tip region of the catheter tip the characteristics of this region can be manipulated. Preferably, these hard and soft areas comprise alternately provided hard and soft rings. The dimensions and number of these rings determine, together with the material that is provided, the characteristics of the catheter region. This provides sufficient freedom to design the catheter according to its needs. For example, by changing the frequency of the hard and soft rings, the flexibility in axial direction of the catheter of this region of the tip can be manipulated.

In a further preferred embodiment according to the present invention during the providing of the catheter the angle of the reinforcement wire in the reinforcement layer is locally adjusted.

By providing a reinforcement layer that is locally provided with a different angle, the flexibility of this reinforcement layer is also locally adjusted. The reinforcement layer comprises SS304 material, for example. The thickness of the wires in the reinforcement layer is 0.01-0.20 mm and have a width of 0.05-20 mm, for example. Other dimensions are also possible depending on diameter and wall thickness, for example. Typical angles for the wires are for guiding catheters 30-80 degrees and for diagnostic catheters 20-70 degrees. Also other angles are possible depending on the specific use, diameter and wall thickness, for example. During a treatment of the catheter in accordance with the invention the braiding wires are kept in place. For example, when performing a heat treatment spools can be used with an opposite field to prevent heating of the wires so that they maintain their desired position.

In a further preferred embodiment according to the present invention during the providing of the catheter locally the top layer is omitted.

By preventing the top layer to be provided on a part of the catheter the actual removal of material is not required. This results in an efficient making of the catheter according to the present invention. This can be achieved by using intermittent extrusion with one material stream, or by shielding sections on top of the reinforcement layer to prevent the forming of a top layer thereon, for example.

In a further preferred embodiment according to the present invention during the providing of the catheter the top layer is prevented to strongly adhere to the other layers of the catheter.

By preventing a strong adherence of a top layer to the reinforcement layer in one or more specific locations of the distal end, the material at these locations can be removed relatively easy. For example, the prevention of a strong adherence can be achieved by applying oil, grease or an alternatively bad adherent solution on top of a reinforcement layer before the top layer is provided. Removing the material at these locations is relatively easy and after cleaning the removed material can be replaced by a different material with the desired characteristics.

### BRIEF DESCRIPTION OF THE DRAWINGS

Further advantages, features and details of the invention are elucidated on basis of preferred embodiments thereof, wherein reference is made to the accompanying drawings:

FIG. 1 is a front view of a catheter according to the present invention;

FIG. 2 is a detail of the tip of the catheter of FIG. 1; and

FIG. 3 is a cross section of the catheter of FIG. 1.

### DETAILED DESCRIPTION OF THE PREFERRED EMBODIMENTS

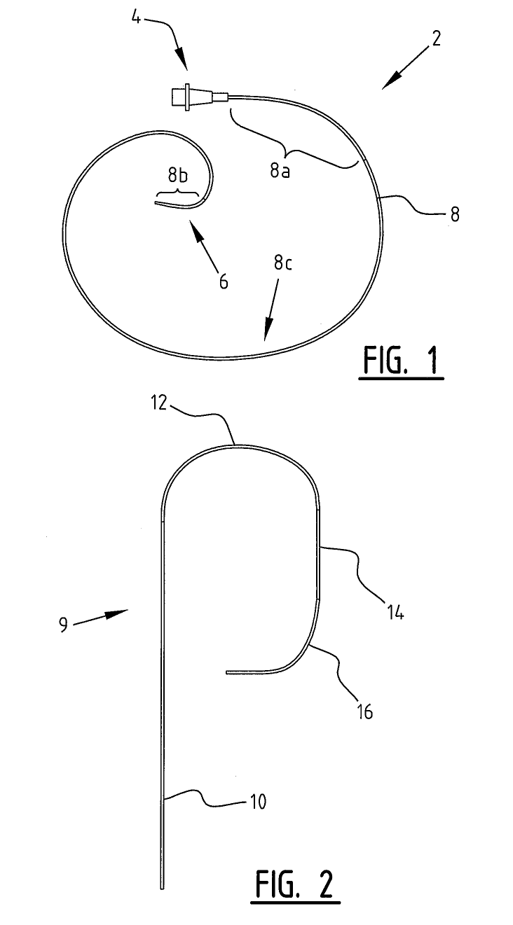

A guiding catheter 2 (FIG. 1) comprises a proximal end 4 and a distal end 6. Proximal end 4 and distal end 6 are connected by tubing 8.

The tubing 8 may have any suitable length. The tubing 8 can be divided in three sections: a proximal section 8a, a distal section 8b and an intermediate section 8c. Of these sections, at least section 8c is provided with a coating such that the friction of this section in use is lower than the friction experienced by proximal section 8a and distal section 8b. In the illustrated embodiment, shown in FIG. 1, the proximal section 8a is uncoated and has a length of approximately 25 cm, the distal section 8b is uncoated and has a length of approximately 7 cm, and the intermediate section 8c is coated with a hydrophilic coating.

The catheter tip 9 (FIG. 2) starts from basic polymer material 10 with a distal tip 12 of a relatively high elastic stiff material. The next part of tip 9 is the intermediate tip 14 of a relatively stiff material. In the illustrated embodiment the stiff material of intermediate tip 14 is similar to the body material of the body part 10. The outer end of tip 9 is soft tip 16 that comprises a soft material without reinforcement, at least not in the illustrated embodiment. For the illustrated embodiment of catheter 2 radial stiffness of body part 10 and distal tip 12 is about 50 N/mm and bending stiffness between 6.0 and 10 N/mm, for the intermediate tip 14 radial stiffness is about 27 N/mm and bending stiffness is about 0.5-4 N/mm, and for the soft tip 16 radial stiffness is about 0.5 N/mm and bending stiffness is about 0.05-0.25 N/mm.

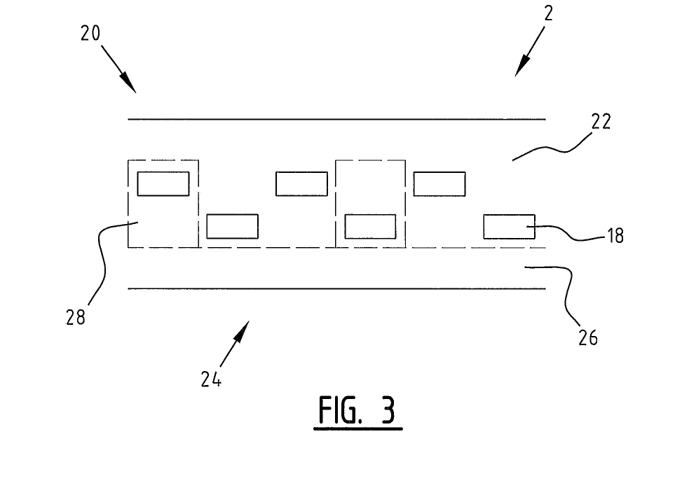

In an embodiment of catheter 2 (FIG. 3) the reinforcement layer comprises braiding 18. Towards the outside 20 of catheter 2 is provided top layer 22. Towards the inside 24 of catheter 2 is provided inside inner layer 26. Material of top layer 22 extends between braiding 18 over a part of the length of catheter 2. Optionally, in case when removing material to enable providing top layer 22 some parts of the former material 28 are kept in place. This provides relatively hard and relatively soft parts of the catheter 2. The outside 20 of catheter 2 is smooth as top layer 22 extends over the entire surface area of catheter 2.

The process for making catheter 2 comprises a number of steps that are known to the skilled person. In addition to these steps locally material of the top layer is removed and/or providing material at these locations is prevented. Specific material realizing the desired characteristics of specifically the tip 9 of catheter 2 replaces the original material.

In use catheter 2 provides an optimal back-up support, especially for the guiding catheter 2, and a shape that can be straightened relatively easy for easy loading of the catheter over the guide wire into the vascular system, optimal alignment of the tip to the local take-off angle of the coronary artery and an improved a-traumatic tip 9 of catheter 2.

It is noted that the tip sections 12, 14, 16 in general will not necessarily coincide with the uncoated/coated sections 8a, 8b, 8c. In general, one or more coating layers will be applied over the top layer 22 of the catheter to provide the sections 8a, 8b, 8c.

The present invention is by no means limited to the above described preferred embodiments thereof. The rights sought are defines by the following claims within the scope of which many modifications can be envisaged. The means and method steps described above can be applied to the distal end, on one or more of the parts or sections thereof.

1. A catheter having a proximal end and a distal end, and comprising from the distal end towards the proximal end:
a distal section;
an intermediate section; and
a proximal section,
wherein the intermediate section is provided with a coating such that the intermediate section experiences less friction in use than the distal section and the proximal section.

2. The catheter according to claim 1, wherein the coating is a hydrophilic coating.

3. The catheter according to claim 1, wherein the distal section has a length of 1-15 cm, preferably 3-15 cm, more preferably 3-10 cm, even more preferably 5-9 cm and most preferably approximately 7 cm.

4. The catheter according to claim 1, wherein the proximal section has a length of at least 3 cm, preferably at least 5 cm, more preferably at least 10 cm, even more preferably at least 20 cm and most preferably at least 25 cm.

5. The catheter according to claim 1, wherein the catheter is a guiding and/or diagnostic catheter.

6. The catheter according to claim 1, further comprising a tip including in the direction from the distal end towards the proximal end:
a soft tip;
adjacent to the soft tip an intermediate tip; and
separated from the soft tip by the intermediate tip a distal tip,
wherein the tips are provided with different characteristics relating to the resistance against bending and/or positioning and/or relaxation of the catheter in a body lumen.

7. The catheter according to claim 6, wherein the tip gradually becomes softer towards the soft tip.

8. The catheter according to claim 6, wherein the distal tip is relatively elastic as compared to the intermediate and soft tips.

9. A method of providing a catheter, comprising providing a coating to an intermediate section of a catheter between an adjacent distal section and an adjacent proximal section of the catheter such that the intermediate section experiences less friction in use than the distal section and the proximal section.

10. The method according to claim 9, further comprising:
providing the catheter with an elongate tube;
at a tip of the catheter locally removing material from and/or preventing material to be provided on the top layer of the tube; and
replacing the removed material with and/or placing a different material.
